(19) United States
(12) Patent Application Publication
(10) Pub. No.: US 2016/0082088 A1
(43) Pub. Date: Mar. 24, 2016

(54) COLLAGEN POWDER

(71) Applicants: Jan Gert TEN KATE, Eelderwolde (NL); Petrus Rimke VISSER, Nijmegen (NL)

(72) Inventors: Jan Gert TEN KATE, Eelderwolde (NL); Petrus Rimke VISSER, Nijmegen (NL)

(73) Assignee: HFP Ingredients B.V., Groningen (NL)

(21) Appl. No.: 14/839,963

(22) Filed: Aug. 29, 2015

Related U.S. Application Data
(63) Continuation of application No. 13/700,583, filed on Mar. 7, 2013, now abandoned.

(30) Foreign Application Priority Data
May 28, 2010 (EP) 10164379.9

(51) Int. Cl. A61K 38/39; A61K 9/16; C07K 14/78; A23L 1/305; A23L 1/29

(52) U.S. Cl. CPC A61K 38/39; A23L 1/305; A23L 1/293; C07K 14/78; A61K 9/16; A23V 2002/00

(57) ABSTRACT

Methods are disclosed for preparing a collagen powder, for example which may be used for the preparation of a satiety inducing food product. The collagen powder is suitable as a food additive in food products for the reduction of hunger and as a possible treatment of obesity and its pathophysiological consequenceos, such as metabolic syndrome.

### CROSS-REFERENCE TO RELATED APPLICATIONS

This application is continuation of co-pending U.S. application Ser. No. 13/700,583, filed Mar. 7, 2013, which is a U.S. National Stage application under 35 U.S.C. §371 of International Application PCT/NL2011/050381 (published as WO 2011/149356 A1), filed May 30, 2011, which claims priority to Application EP 10164379.9, filed May 28, 2010. Benefit of the filing date of each of these prior applications is hereby claimed. Each of these prior applications is hereby incorporated by reference in its entirety.

### FIELD OF THE INVENTION

Aspects of the invention relate to a method for preparing a collagen powder and to collagen powder that can be used for the preparation of a satiety inducing food product. The collagen powder may be suitable as a food additive in food products for the reduction of hunger and as a possible treatment of obesity, which is the main risk factor for the development of metabolic syndrome.

### BACKGROUND OF THE INVENTION

Collagen is the major fibrous protein in animals. It is probably the most abundant animal protein in nature. Collagen has a molecular weight of about 300 000 Dalton, and is a combination of three polypeptide strands of the triple helix, wherein each strand is coiled into helical structure. Collagen comprises of a large number of fibres which, in turn, further comprise of a much greater number of fibrils of submicroscopic size. The fibrils have a diameter in the order of 1-5 nm and lengths ranging from several hundreds up to hundred thousands of nm.

If collagen is partially hydrolysed, the three polypeptide strands of the collagen molecules (tropocollagen strands) separate into globular, random coils, producing gelatine, which is used in many foods, including flavoured gelatine desserts. Besides food, gelatine has been used in pharmaceutical, cosmetic, and photography industries.

Due to the ever increasing prevalence of obesity world-wide and the associated diseases, much attention has been focused on strategies to combat the problem. Such strategies for weight management are directed to intensifying the metabolism, inhibit digestion and/or reducing calorie and food intake.

One type of food product that is applicable in the reduction of calorie intake are food additives that induce a satiety effect more quickly and reduce the appetite. This effect of satiety arises due to food intake and persists until the stomach is emptied. Due this effect, a person no longer has an appetite and is less likely to consume more food. Therefore food products that persist longer in the stomach and are slowly digested have a longer lasting satiety effect. Recent interest has focused on collagen material, due its satiety inducing properties, as being a suitable additive in weight loss food products.

WO-A-2009/008714 describes that collagen hydrolysate can be used for the preparation of an edible composition for limiting voluntary food intake. The preferred average molecular weight of the collagen hydrolysate is described as being between 2 and 10 kDalton. The collagen hydrolysate is described as being obtained by controlled hydrolysis of gelatin which is obtained from animal collagen. Possible sources of animal collagen are not specifically described.

WO-A-2005/023017 describes a food composition comprising from 5 to 30 wt. % hydrolysed gelatin and a tryptophan source which is suitable to be used in meal replacement products. The preferred average molecular weight of the hydrolysed gelatin is disclosed as being from 2 to 10 kDalton. The hydrolyzed gelatin is said to be able to be prepared by enzymatic (proteolytic) treatment and to have different properties on its source and method of production.

EP-A-1 471 802 describes a satiety inducing food product, consisting of a cross-linked protein powder, that is subject to a reduced digestion speed in the stomach. The cross-linked powder is described as being obtained by cross-linking a protein into a gel, wherein said gel is dried. The preferred protein is described as being gelatin.

Collagen can be recovered from animal hide or skin, in particular from dehaired hide splits, such as bovine or from skins, such as porcine and poultry. A split refers to the inner or under layer of a hide or skin.

Several methods for the recovery of collagen from animal skin have been described in the prior art. EP-A-1 270 672 describes a process for recovering native collagen wherein hide or skin is comminuted in an aqueous suspension, liberated fat is separated, the pH of the suspension is made alkaline, a protease which does not degrade the native collagen is added, the suspension is acidified, the native collage is separated off and resuspended in water for disinfection and further purification, and finally the purified and disinfected native collagen is separated off as a solid phase. Collagen in the form of a powder is not disclosed.

U.S. Pat. No. 3,932,677 describes a process for preparing an edible collagen from a limed hide collagen source, wherein hide collaged source is delimed by treatment with a dilute, edible acid at a pH of about 4.0 to 5.5, washing water soluble calcium salts from the hide collagen with water, forming an extrudable slurry from the neutralised and washed hide collagen and extruding said slurry through an annular die to form a tubular edible collagen casing.

WO-A-92/10944 describes a method in which raw hide splits are washed in dilute acid and one or more times with water to neutralise and remove the alkali, such as sodium sulphide and lime which are conventionally used to remove hair and residual fat. Before or after washing the hide splits are cut into pieces with typical dimensions of about 5-10 mm by 3-5 mm. These pieces are thereafter dried in a two stage drying process, wherein the first stage involves drying in a first stream of heated gas maintained at a temperature of less than about 60° C., and the second stage involves drying in a second heated gas stream at a temperature which is higher than said first stream, wherein the temperatures and treatment times during the drying are controlled to minimize discoloration and hydrolysis of the dried product. Finally, the dried collagen is milled to form a fibrous product. Disadvantages of this product include that the collagen is hard to wet, the collagen has a low Bloom gel strength, and the bulk density of the collagen is low.

Other processes for producing collagen products are for instance known from U.S. Pat. No. 5,411,887, US-A-2005/267292 and U.S. Pat. No. 5,484,888.

### SUMMARY OF THE INVENTION

Aspects of the disclosure relate to processes for preparing a dry collagen powder.

Other aspects relate to providing a collagen powder having improved satiety inducing properties for use in the preparation of food products.

Further aspects relate to providing a collagen powder having improved gel strength.

Yet further aspects relate to providing a collagen powder which can suitably be used as an emulsifier.

Still further aspects relate to providing a collagen powder which can suitably replace so-called “functional ingredients” used in foodstuffs, such as soy protein, soy isolates, caseinate, fosfates, alginates or the like. The inventor surprisingly found that this object can be met by a collagen powder having a specific Bloom gel strength.

Additional aspects relate to providing a collagen powder, viz. a dried product, that has not degraded or has degraded substantially less than known collagen powders. In accordance with some embodiments, the triple helix forming the collagen molecule is untangled resulting in individual tropocollagen molecules, which make up the powder. Such a collagen powder was found to have superior properties compared to known collagen powders, as expressed for instance by its high Bloom gel strength.

### BRIEF DESCRIPTION OF THE DRAWINGS

FIG. 1 illustrates the reduction in food intake, relative to that of a normal control diet, caused by the provision of a high protein diet.

FIG. 2 illustrates a very sharp reduction in food intake in the first two hours (CT 12-14) after the food was returned due to addition of collagen.

FIG. 3 illustrates the increased 24 hour water intake measurement in the CC diet.

FIG. 4 shows that the addition of collagen results in a significant reduction in body weight over the longer term.

FIG. 5 shows the resulting insulin profiles of the blood during the IVGTT.

FIG. 6 depicts the GPC pattern in which more than 99 wt. % of the particles have a molecular weight of about 80 kDa.

### DETAILED DESCRIPTION OF THE INVENTION

It was found that the desired properties of the collagen powder can be obtained by first converting hides or skins to a wet fibrous mass at a moderate temperature of 50° C. or less, followed by drying the wet fibrous mass at a high temperature, which is realized by contacting the wet fibrous mass with a contact dryer having a surface temperature of preferably 150° C. or higher. The low temperature of the first, wet stage is kept low, in order to minimize the hydrolysis of the collagen proteins. Also no enzymatic additions are required in this stage. This makes the process of the present invention fundamentally different from known processes, such as EP-A-1 270 672 and U.S. Pat. No. 5,411,887. Subsequently the fibrous mass is dried at a high temperature of 150° C. or more. Surprisingly during the drying no, or only limited hydrolysis of the collagen material occurs, so that the dried collagen material is still composed of relatively large molecules, which explains the superior quality of the product of the present invention.

Accordingly, in a first aspect the invention is directed to a process for producing collagen in the form of a powder, which process comprises first producing a wet collagen product (fibrous mass) from a hide or skin at a temperature of 50° C. or less, followed by drying said wet collagen product at a contact dryer, which typically has a surface temperature of 150° C. or higher, to obtain said collagen powder. The wet collagen product is obtained typically by subjecting the hide or skin to process steps such as a size reduction step; an alkaline and/or an oxidizing treatment step; and a neutralizing step following said alkaline and/or an oxidizing treatment.

In a further aspect, the present invention is directed to a collagen powder, having a Bloom gel strength of 300 g or more as determined by standard test methods, in particular by ISO 9665. The “Bloom gel strength” is a measure of the ability of a material to form a gel. The Bloom gel strength is the weight in grams required to depress the gel a distance of 4 mm with a piston having a cross-sectional area of 1 cm², the gel first having been cooled for a defined time under defined conditions. Thus, the higher the Bloom gel strength of a material is, the greater the ability of that material to form a gel. The gel for this measurement has a standard concentration of 6.67% and has been kept 17 hours at 10° C. prior to measurement.

It was found that such a collagen powder according to the invention has advantageous hygroscopic qualities.

The collagen powder of the present invention is characterized by a very narrow distribution in molecular weight. It was found that 99 wt. % or more of the collagen particles has the same molecular mass. This may for instance be measured using gel permeation chromatography (GPC). FIG. 6 shows the result of a typical GPC measurement on a collagen powder according to the present invention, in which a single peak is seen, indicative for a narrow distribution in molecular weight. Thus in accordance with the present invention the collagen molecules have a very narrow molecular weight distribution. Also the collagen molecules have a molecular weight that is considerably higher than collagen obtained according to prior art methods.

The collagen powder of the present invention has excellent heat stability, which makes it suitable for addition to food products that need to undergo subsequent heat treatment, e.g. pasteurization. Surprisingly the collagen powder of the present invention maintains its functionality with respect to emulsifying and water binding properties when subjected to such heat treatments.

Surprisingly, the collagen powder of the invention was found to have improved satiety inducing properties when used as a food supplement, compared to known products used for this purpose.

The excellent properties of the collagen powder of the invention are believed to be the result of the high nativity of the collagen. This means that the protein molecules making up the collagen powder are relatively large compared to collagen obtained by conventional processes. In a preferred embodiment, the collagen powder of the invention is of bovine origin and comprises particles having a molecular mass of about 80 kDa. Preferably more than 99 wt. % of the particles, more preferably about 100% of the particles have a molecular weight of about 80 kDa, as may be measured by using GPC, for instance resulting in the GPC pattern depicted in FIG. 6.

The collagen powder of the invention is suitable for the use as a food additive in food products for the reduction of hunger and weight management. It can thus be used as a possible treatment of obesity.

For good satiety inducing properties it is preferable that the food products comprises per dosage (i.e. unit to be consumed) at least 7 gram collagen powder of the invention and preferably less than 15 gram wt. %. Typically a food composition according to the present invention comprises 2 to 4 wt. % (based on dry weight) of the collagen of the invention.

Examples of suitable food products in which the collagen powder of the invention can be applied include but are not limited to the following: tablets, snacks, nutritional bars, ready made meals, baked products (e.g. muffins), meat products, instant soups, reconstitutable powder drinks, soups, drinks, yoghurts and health shakes.

The ingredients for such food products would be dependent on the type of food product. However typical ingredients can comprise such components as vegetable, fruit, eggs, meat or milk products, seeds, grains, pulses, fats or oils and water. The food product may also further comprise aromatic substances, colorings and flavorings, food salts, bulking agents, emulsifiers, saccharides, artificial and natural sweeteners such as sugars and sugar sources, carbohydrates, vitamins, alginates (e.g. sodium alginate) and minerals.

It was also found that such collagen powder has advantageous emulsifying properties. Furthermore, the collagen powder of the invention was found to be able to improve the structure of food products in which it is used. A relatively small addition, in the order of 1%, will significantly increase of the viscosity of meat emulsions. Substitution of phosphates in meat products by collagen powder without loss of thermal stability is another option.

Moreover, the collagen powder was found to be a suitable replacement of soy protein. This is advantageous in view of the growing demand for clean-label food products.

Collagen powder of the invention has a Bloom gel strength of at least 300 g, preferably at least 350 g, more preferably at least 400 g, and even more preferably at least 450 g. These Bloom gel strengths are superior than those mentioned in the prior art for other collagen products. The upper limit of the Bloom gel strength of the collagen powder of the invention can be high as 550 or even 600 g.

Preferably the collagen powder of the invention is a bovine collagen powder, in particular a powder comprising less than 1 wt. % fat (dry matter basis) and at least 95 wt. % protein (dry matter basis; as determined by N×5.52 method. For porcine and poultry collagen powder of the present invention the fat percentage is generally higher, e.g. 5-20 wt. %. Protein contents are typically 80 wt. % or more.

Unlike gelatin, the collagen powder of the present invention does not give a clear solution in water. It is preferred that the collagen powder of the invention has a powder structure, in particular a free flowing powder.

Preferably, the collagen of the invention has a high number average molecular weight, e.g. around 80 kDa for bovine collagen. For collagen of porcine or poultry origin, similar values of around 80 kDa are obtained.

The collagen powder of the present invention has remarkable emulsifying properties. For instance, using as little as 1 part of powder to 75 parts of water and 75 parts of oil or animal fats in a results in a stable warm emulsion (>40° C.). Using vegetable oils a stable cold system (viz. less than 10 ° C.) can be obtained with as little as 1 part powder to 60 parts water and 60 vegetable oil.

The collagen powder of the invention gels already in a 1:15 ratio (wt./wt.) with cold water (<10° C.). Gelling in cold water can be achieved in relative amounts of down to 5 wt. % collagen or even less. In hot water (>40° C.) the collagen gel will may already form a firm gel in relative amounts as low as 3.5 wt. % or even down to 2.5 wt. %.

The collagen of the invention is remarkably heat stable. It will reform to a gel even after pasteurization/sterilization, which makes the protein very suitable for use in food products, in particular in canned, sterilized or pasteurized food products.

By virtue of its exceptionally high Bloom gel strength, the collagen powder of the invention can be advantageously used as a gelling agent. The collagen powder of the invention was found to keep a high bloom count (typically more than 225) even after heating to 100° C.

In a further aspect the invention is directed to a method for preparing a collagen powder. This method can be used to obtain the collagen powder described herein.

In the method of the invention, an animal hide or skin is subjected to a first stage size reduction, for example by using a standard meat grinder, bowl cutter, shredder or the like. This may be followed by a second stage size reduction in which the size of the skin or hide particles is further reduced. This step is followed by a step of dispersing the milled skin or hide in water, preferably in a stirred tank and then subjecting the milled skin or hide to an alkaline and/or oxidizing treatment. After this step, the pH of the dispersion is neutralized, viz. adjusted to about 5-7.

Next, the solids may be separated from the liquid, for example by centrifugation and/or using a screen. Then a third stage size reduction step may be carried out, which is followed by redispersing the fibrous mass in water. This may be followed by isolating the solids, for instance by centrifugation and/or screening. In accordance with the present invention this wet stage of the process the process is carried out at the low temperature of 50° C. or less, preferably below 45° C., even more preferably at 42±2° C.

The wet stage is followed by the drying stage, which is carried out at the relatively high temperature. According to the invention this high temperature is obtained by means of a contact drying step, which assures relatively short contact times. In this high temperature drying step the surface temperature of the contact dryer is preferably kept at 150° C. or more, preferably 160° C. or more, more preferably 165±4° C. The drying may be followed milling and screening to obtain a powder product having the required particle size.

In one typical embodiment, the method of the invention comprises:
providing animal hide or skin;
size reduction in one or more steps;
dispersing the particles in water;
subjecting the dispersion to alkaline treatment and/or to oxidizing treatment, which results in the dispersion of hide or skin particles being converted into a fibrous mass;
adjusting the pH of the fibrous mass to neutral (7) or slightly acidic pH;
separating the solids and the liquid contained in said fibrous mass;
subjecting the solids to a further size reduction step;
dispersing the solids in water;
again adjusting the pH to neutral or slightly acidic pH;
again separating the solids and the liquid in said fibrous mass; and
size reduction and/or particle separation to obtain a product having the desired particle size distribution.

Optionally a metal detection step is carried out before or during one of the first size reduction steps.

During the entire wet part of the method the temperature is maintained at a level so that the native collagen will not or only to a very small extent degrade, denature or gelatinize. The temperature depends to an extent on the type of collagen treated, but it should preferably not be greater than 50° C. More preferably the temperature is not greater than 45° C. and even more preferably no greater than 40° C. However, in one or more subsequent drying steps the temperature can be increased to much higher temperatures. One would expect that higher temperature would result in fragmentation of the collagen molecules. However the present inventors found that by using a contact dryer, contact time can be kept sufficiently short: in order of seconds to several minutes, e.g. from 10 seconds −5 minutes, preferably 15 seconds to 2 minutes.

Thus the method of the present invention surprisingly allows the preparation of collagen powder having exceptionally high Bloom gel strength, as well as exceptional emulsifying and heat stability properties.

The applied animal hide or skin is preferably hide split or fresh skin. It can originate from various animals such as bovine, porcine and poultry and is preferably from bovine. Bovine hide splits that are limed are advantageous, because they have a relatively low fat content, typically less than 10 wt. % based on dry solids with a pH value from 10 to 12. Fresh porcine and poultry skins have a higher fat content, typically about 20-40 wt. %, based on the raw materials and have a high plate count.

The animal hide or skin is pre-ground in a first stage size reduction using a commercial grinder, such as a Wolfking™ or Meatwolf™ grinder. Preferably this results in ground hide or skin having an average size of several millimeters, for instance having a largest diameter of 2 to 20 mm, e.g. strips measuring 3-4 by 10-15 mm.

The ground animal hide or skin may then be subjected to metal detection, e.g. based on induction or magnetism. In a second stage size reduction, the animal hide or skin may be subjected to fine grinding typically to sizes of about 2-5 mm, e.g. 1-2×3-5 mm, using again for instance Wolfking™ or Meatwolf™ grinders.

The animal hide or skin is subjected to an alkaline treatment and/or an oxidizing treatment. The alkaline treatment is applied in order to remove residual fat. Usually, the alkaline treatment involves the application of an aqueous solution comprising an alkaline agent such as sodium hydroxide or calcium hydroxide. Preferably is the alkaline agent calcium hydroxide. Typically the concentration of calcium hydroxide used is from 1 to 2% and is preferably 2%. The pH during the alkaline treatment can be at least 10, preferably at least 11, more preferably at least 12. Preferably the pH during the alkaline treatment is no greater than 12.5. Since the bovine hide splits are already limed, only the fresh porcine and poultry skins are treated. The alkaline treatment can be performed very fast, e.g. within several seconds to minutes, e.g. typically less than 10-20 seconds.

The oxidizing treatment is applied in order to decontaminate the animal hide or skin, particularly the fresh porcine and poultry skins. In limed bovine hide splits, the treatment also oxidizes residual sulphide from the tanning process to suphite/sulphate. This prevents the production of hydrogen sulphide upon pH reduction in the neutralizing treatment. The oxidizing treatment is an aqueous solution comprising an oxidizing agent such as hydrogen peroxide or ozone. Preferably is the oxidizing agent hydrogen peroxide. Typically the hydrogen peroxide concentration is about 300 to 2500 ppm. The oxidizing treatment can be performed for several seconds to minutes, typically less than 10 seconds. The oxidizing treatment is preferably applied together with the alkaline treatment.

Following this, the resulting product is subjected to a pH adjustment to bring the pH at a neutral or slightly acidic pH 5-7. This can be done by acidifying the dispersion. This can involve washing the skin or hide in an aqueous solution of an acid. The purpose of neutralizing is, among others, the deliming of the skin or hide. During the neutralizing treatment water soluble calcium salts are removed from the hide. Preferred acids for neutralizing the hide or skin include lactic acid, hydrochloric acid, carbon dioxide, acetic acid, ethylene diamine tetraacetic acid, ammonium chloride, propionic acid, and fumaric acid. More preferably is carbon dioxide or acetic acid the preferred acid.

The pH should preferably not be lower than 4.2. It was found that optimal results are obtained if pH is kept between 5-6. The neutralizing treatment can be carried out very quick and can be completed in e.g. several seconds to about one minute.

The animal hide or skin is then subjected to a step for separating slits from the liquid, e.g. decanted or tri-canted. Following this, the animal hide or skin is again decanted or tri-canted to separate out the solids. The bovine collagen dry matter of the solids is typically between 10 to 30%. The porcine and poultry collagen dry matter is typically 10 to 35% and further comprises 2 to 7% fat.

The neutralized animal skin or hide may then be subjected to a third stage size reduction where it is finely ground. As an optional step, the wet, ground porcine and poultry skin is isolated as a final product. It is a microbiologically stable product, comprising of a protein and fat content of about 20 and about 4 wt. % respectively.

Thereafter the ground product must be dried. Drying is preferably carried out using a contact dryer, in particular a roller dryer (or drum dryer). Other contact dryers are belt dryers or plate dryers. Using a drying roller, as well as drying the animal hide or skin after grounding, is advantageous in view of moisture transport. Because these measures provide excellent moisture transport, it suffices in general to dry the ground animal hide or skin on the roller dryer for a time period of 2 minutes or less, or even 1 minute or less, e.g. 10 to 60 seconds.

Because the drying is carried out at a relatively high temperature, this step can be performed quickly, which adds to process economy. In addition these short contact times are believed to add to maintaining the high molecular weight and narrow distribution in molecular weight of the product.

If the collagen is dried by means spin flash drying or air drying, this does not result in a dried product having the desirable properties.

As a final step, the collagen product is milled or sieved (screened). The milled and/or screened animal hide or skin can have an average particle size of 200 μm or less, preferably 100 μm or less. In a preferred embodiment, the average particle size of the ground animal hide or skin is in the range of 10-100 μm.

It was surprisingly found that storage of the ground animal hide or skin before drying has a detrimental effect to the Bloom gel strength of the resulting collagen powder. Without wishing to be bound by theory, the inventor believes that micro-organisms play a role in this degradation of the product. It is therefore preferred that the ground animal hide or skin is dried within 24 hours after grinding, preferably within 12 hours, more preferably within 6 hours, and most preferably directly after grinding.

Optionally, the dried collagen powder can be size separated, for instance by using one or more suitable sieves.

The method of the invention provides a collagen powder that has a substantially non-fibrous structure. In addition, the collagen powder has a relatively high bulk density of at least 540 g/l, preferably at least 550 g/l.

The invention is now elucidated on the basis of some examples, which are not intended to limit the scope of the invention.

### EXAMPLES

Viscosity Experiments

A 10% gelatin solution was made according to EP-A-1 471 802. The gel solution was cross linked using transglutaminase (Active EB from Ajinomoto) at pH 6 for 24 hr at 40° C. A firm mass formed that remained firm at a temperature at 40° C. This in contrast to an untreated 10% gelatin solution which is liquid at 40° C.

A 5 g piece from the cross linked gelatin was transferred to a 100 ml solution further comprising one Betaine/Pepsine pill (from AOV Orthomolecular Food Supplement BV). The pH and temperature were 2.5 and 37° C., respectively and were maintained for the duration of the experiment. After four hours, there was no detectable degradation of the gelatin. However this is not a realistic simulation of what happens in one stomach, crosslinked gelatin would be dry ground and possibly added to a food as a supplement. It would not be consumed as a big chunk.

100 g of crosslinked gelatin was resuspended in 100 ml water containing a Betaine/Pepsine pill in a blender at a pH of 2.5 and temperature of 37° C. The viscosity of the suspension was measured with a Brookfield viscosimeter using a spindle 3 and a rotational speed or 50 rpm. At t=0 min the viscosity was 1200 cp, after 30 min the viscosity was <40 cp and the gelatin was completely liquid. From this result we should not expect that the product of EP-A-1 471 802 to be very effective as a hunger suppressant.

A similar test was performed using Caseinate EM7 from DMV and the collagen powder of the invention, Kapro 95. In two separate solutions, each containing a Betaine/Pepsine pill, 20 g EM7 and 20 g Kapro 95 were suspended respectively. The solutions had a pH value of 2.5 and a temperature of 37° C. Viscosity measurements were made with a Brookfield viscosimeter using a spindle 3 and a rotational speed or 50 rpm. The results are shown below (see table 1).

TABLE 1

| | Viscosity | Viscosity | Viscosity | Viscosity |
|---|---|---|---|---|
| Sample | (t = 0 min) | (t = 60 min) | (t = 120 min) | (t = 180 min) |
| EM7 | 1300 cp | 600 cp | 500 cp | 400 cp |
| Kapro 95 | 8300 cp | 6000 cp | 4000 cp | 4200 cp |

The results show that Kapro 95 remains highly viscous for a longer period time than EM7.

### Example 1

### Short Term Effect of Bovine Collagen

The Example was performed using three groups of rats, comprising eight animals per group. One group received the standard rat diet (lab chow) and served as a general control. The experimental groups received a combination of 60% lab chow and 40% bovine collagen according to the invention (Collagen Care, abbreviated to CC). There was also a third group, who received a combination of 60% lab chow supplemented with 40% casein. This third group served as controls for the high protein diet of the experimental CC. During the experiments, the animals had food available for 20 hours a day.

Circadian Time, or CT, is a way to experimentally compare day and night activities of animals. In a 12 hours dark / light regime, CT 0-12 was defined as the inactive period (in humans is that the night, in rats is that the light period). CT 12-24, was conversely defined as the active period (in humans the light, in rats the dark). This was the experimental method used.

The animals were housed individually for three days and were food intake, water intake and changes in body weight measured. The exact experimental protocol was as follows:
Circadian time CT 8: food is removed +body weight measured
Food is returned on CT 12, food and water intake measured from CT 12 - CT 14 (meal two hours) and for CT 12 - CT 8 (food intake over 24 hours is determined.)

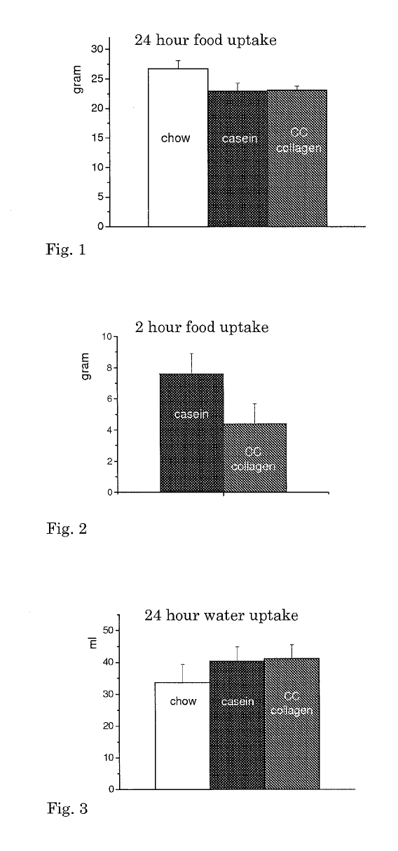

The provision of a high protein diet caused a reduction in food intake to about 85% of that of the normal control diet (see FIG. 1). This effect is often seen in diets with a high protein content and is comparable with the effect observed of the popular Atkins diet from the USA. The satiating effect of the high protein diet resulted in the space of three days in a light but significant weight loss (5%) compared to the control diet. This applied for both the CC diet and the casein diet.

Addition of bovine collagen led especially to a very sharp reduction in food intake in the first two hours (CT 12-14) after the food was returned (see FIG. 2). This was the most striking effect of the CC diet compared to the casein diet (and of course compared to the control diet).

Also the 24 hour water intake measurement was increased in the CC diet (see FIG. 3). The two hour water intake (CT 12-14) was not significantly different between the CC group and the control group. However if one looks at the so called prandial drinking (water intake per amount of food), then the CC animals drink relatively more.

### Example 2

### Long Term Effect of Bovine Collagen

The Example was performed using four groups of rats, comprising eight animals per group. All animals received a standard lab chow diet enriched with either 20% or 40% protein. Two groups received bovine collagen according to the invention (CC), while the other two groups received casein. The four groups consisted of:
CC 20: a combination of 80% lab chow and 20% bovine collagen
CC 40: a combination of 60% lab chow and 40% bovine collagen
20 casein: a combination of 80% lab chow and 20% casein
40 casein: a combination of 60% lab chow and 40% casein
   The casein groups served as controls for the high protein diet comprising of bovine collagen. During the experiments, the animals had food available ad lib.

The animals were housed individually and during a period of 14 days, measurements were taken twice daily at CT 9 was the 24-hour of food intake, water intake and changes in body weight.

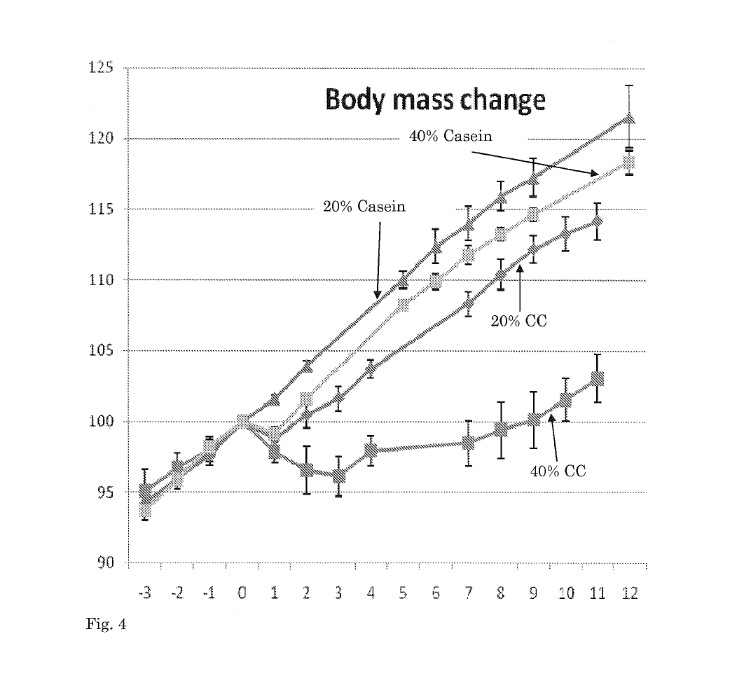

The results from the experiment were that the addition of bovine collagen reduced food intake and increased water intake. The data of the food and water intakes calculated cumulatively over 14 days were significantly different. FIG. 4 shows the body weights over the first period. It is clear from FIG. 4 that the addition of bovine collagen results in a significant reduction in body weight over the longer term.

Both short and long term food intake in rats was greatly reduced by the consumption of diet enriched with bovine collagen according to the invention (CC). This is possible due to a combination of two properties of the CC diet, namely (a) the high protein content, and (b) a high Bloom number. Ultimately this leads to an increased stomach filling, inhibited gastric emptying and delays the absorption of food in the blood. This combination creates a highly reduced short term food intake leading to improved insulin profiles in the blood.

In the long term it appears that the bovine collagen according to the invention has an effect of food intake being reduced, leading to a significant reduction in body weight. The addition of bovine collagen in the normal diet has thus a reducing effect of food intake and body weight which means that this diet can potentially contribute to fighting and preventing obesity and its pathophysiological consequences, such as type 2 diabetes in humans and animals.

Finally, it should be noted that both the short and long term effects are very promising. The satiation effect is similar to that from an injection with a recognized satiety hormone like cholecystokinin. Addition of bovine collagen according to the invention to a regular diet appears to offer possibilities in the treatment of obesity and its pathophysiological consequences, such as metabolic syndrome.

### Example 3

### Improved Insulin Profiles in the Blood in an Intravenous Glucose Tolerance Test (IVGTT)

Rats were divided in two groups. The animals were housed individually during a period of 21 days. One group received a diet of 70% lab chow and 30% casein (N=4) and the other group (N=4) received a combination of 70% lab chow and 30% bovine collagen according to the invention (CC). During the IVGTT the rats were infused with 15 mg/min glucose over a 30 minutes period. Before the onset of the infusion, two baseline samples were taken at time points t=−11 and −1 min. After the start of the infusion at t=0 min, blood samples were taken at time points t=5, 10, 15, 20, 25, 30, 40 and 50 min.

Plasma insulin was analysed by radioimmunoassay.

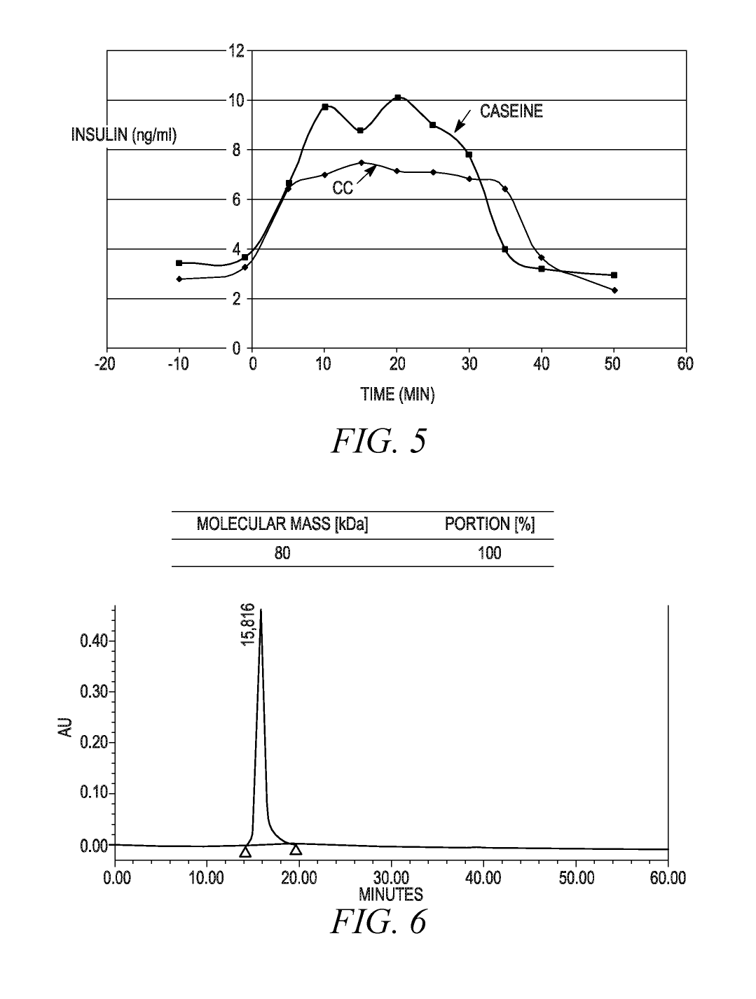

FIG. 5 shows the resulting insulin profiles of the blood in both groups during the IVGTT. In the CC fed rats, the invention caused a significant (p<0.05) reduction in the areas under the curve (AUC) of insulin during the IVGTT.

It can be concluded that that the addition of bovine collagen to the normal diet results in improved insulin profiles in the blood which means that it may contribute to preventing type 2 diabetes in humans and animals.

What is claimed is:

1. A method for preparing collagen powder, the method comprising:
producing, at a temperature of 50° C. or less, a wet collagen product by subjecting a poultry hide or skin to a size reduction; an alkaline and/or an oxidizing treatment; and a neutralizing treatment; followed by drying of said wet collagen product using a contact dryer having a surface temperature of more than 150° C., to obtain said collagen powder.

2. The method of claim 1, wherein the temperature of the steps for producing the wet collagen product is maintained at 45° C. or less.

3. The method according of claim 2, wherein the temperature of the steps for producing the wet collagen product is maintained at 40° C. or less.

4. The method of claim 1, wherein said drying of said wet collagen product is carried out at a surface temperature of said contact dryer of 155° C. or greater.

5. The method of claim 4, wherein said surface temperature of said contact dryer is 160° C. or higher.

6. The method of claim 5, wherein said surface temperature of said contact dryer is 165±4° C.

7. The method of claim 1, wherein said alkaline and/or oxidizing treatment is performed at a pH value of 10 or greater.

8. The method of claim 7, wherein said alkaline and/or oxidizing treatment is performed at a pH value of 11 or greater.

9. The method of claim 8, wherein said alkaline and/or oxidizing treatment is performed at a pH value of 12 or greater.

10. The method of claim 1, wherein the neutralizing treatment results in a pH value of 6 or less.

11. The method of claim 10, wherein the neutralizing treatment results in a pH value from 5 to 6.

12. The method according to claim 1, wherein the size reduction is carried out by grinding to produce a ground poultry hide or skin, and wherein the ground poultry hide or skin is dried within 24 hours after grinding.

13. The method of claim 12, wherein the ground poultry hide or skin is dried within 12 hours after grinding.

14. The method of claim 13, wherein the ground poultry hide or skin is dried within 6 hours after grinding.

15. A collagen powder obtained by the method of claim 1 and having a Bloom gel strength of at least 300, as determined by standard test method ISO 9665 using a gel having a concentration of 6.67% that has been maintained for 17 hours at 10° C. prior to measurement.

16. The collagen powder of claim 15, wherein more than 99 wt. % of the collagen molecules have the same molecular mass.

17. A method for reducing hunger and/or managing weight of a subject, the method comprising administering to the subject the collagen powder of claim 15 in a food product.

18. The method of claim 17, wherein the step of administering the collagen powder is for treating obesity and metabolic syndrome.

19. The method of claim 17, wherein the step of administering the collagen powder is for treating insulin resistance.

20. A food product having a strong satiety effect, the food product comprising up to 5 wt. % of the collagen powder of claim 7, based on dry weight.

21. The food product of claim 20, comprising 2-4 wt. % collagen powder, based on dry weight.

22. The food product of claim 21, comprising 3±0.5 wt. % collagen powder, based on dry weight.

23. The food product of claim 20, wherein said food product comprises an additional ingredient selected from the group consisting of a vegetable, a fruit, an egg, a meat or milk product, a seed, a grain, a pulse, a fat or oil, water, an aromatic substance, a coloring or flavoring, a food salt, a bulking agent, an emulsifier, a saccharide, an artificial or natural sweetener, a carbohydrate, a vitamin, a mineral, and combinations thereof.

24. The food product of claim 23, wherein the natural sweeteners are sugars or sugar sources.
